(19) United States
(12) Patent Application Publication
(10) Pub. No.: US 2008/0249584 A1
(43) Pub. Date: Oct. 9, 2008

(54) METHOD AND DEVICE FOR CARDIOSYMPATHETIC INHIBITION

(75) Inventor: Elizabeth Swanhild Scheurer, Minneapolis, MN (US)

Correspondence Address: SCHWEGMAN, LUNDBERG & WOESSNER, P.A., P.O. BOX 2938, MINNEAPOLIS, MN, 55402, US

(73) Assignee: CARDIAC PACEMAKERS, INC., ST. PAUL, MN (US)

(21) Appl. No.: 11/696,923

(22) Filed: Apr. 5, 2007

(51) Int. Cl. A61N 1/362

(52) U.S. Cl. 607/14

(57) ABSTRACT

Described herein are methods and devices that utilize electrical neural stimulation for inhibiting cardiosympathetic activity in order to reduce the risk of sudden cardiac death. In one embodiment, one or more stimulating electrodes are disposed near the left stellate ganglion. An implantable pulse generator delivers neural stimulation to the electrodes in a manner that inhibits activity of the ganglionic tissue.

### FIELD OF THE INVENTION

This patent application pertains to methods and apparatus for the treatment of disease with electro-stimulatory therapy.

### BACKGROUND

Sudden cardiac death (SCD) refers to an abrupt loss of cardiac pumping function and is one of the most common causes of death due to cardiac disease. It is believed that the usual sequence of events leading to SCD is the degeneration of ventricular tachycardia (VT) into ventricular fibrillation (VF). Both VT and VF are abnormal ventricular tachyarrhythmias that occur when re-entry of a depolarizing wavefront in areas of the ventricular myocardium with different conduction characteristics becomes self-sustaining or when an excitatory focus in the ventricle usurps control of the heart rate from the sino-atrial node. The result is rapid and ineffective contraction of the ventricles out of electromechanical synchrony with the atria. Most ventricular rhythms exhibit an abnormal QRS complex in an electrocardiogram because they do not use the normal ventricular conduction system, the depolarization spreading instead from the excitatory focus or point of re-entry directly into the myocardium. Ventricular tachycardia is typically characterized by distorted QRS complexes that occur at a rapid rate, while ventricular fibrillation is diagnosed when the ventricle depolarizes in a chaotic fashion with no identifiable QRS complexes. Both ventricular tachycardia and ventricular fibrillation are hemodynamically compromising, and both can be life-threatening. Ventricular fibrillation, however, causes circulatory arrest within seconds and is the most common cause of SCD. Preexisting coronary artery disease and its consequences (e.g., acute myocardial ischemia, scarring from previous myocardial infarction, heart failure) are present in most SCD victims. Other factors that may lead to SCD are dilated non-ischemic and hypertrophic cardiomyopathies, congenital heart defects, and genetically determined ion channel anomalies (e.g., long QT syndrome).

Cardiac rhythm management devices known as implantable cardioverter/defibrillators (ICDs) have been shown to be successful in reducing the incidence of SCD. ICDs are implanted in a patient and deliver a shock pulse to the heart when the device detects a tachyarrhythmia such as VT or VF. Such shock therapy, delivered as a cardioversion shock (an electrical shock delivered to the heart synchronously with the QRS complex to terminate VT) or a defibrillation shock (an electrical shock delivered without synchronization to the QRS complex to terminate VF), terminates a tachyarrhythmia by depolarizing all of the myocardium simultaneously and rendering it refractory.

It is also believed that the sympathetic nervous system has a major role in triggering the tachyarrhythmias responsible for SCD. Increased sympathetic tone resulting from myocardial ischemia, for example, is thought to facilitate the transition from VT to VF. This is the rationale for other treatments designed to reduce the risk of SCD that work by blocking the action of the sympathetic nervous system on the heart. For example, pharmacological agents that block adrenergic receptors in the heart (beta-blockers) are commonly used in patients at risk for SCD. Beta-blockers are not always effective, however, and there are side-effects that some patients may find intolerable. Another treatment for reducing cardiosympathetic activity is surgical sympathetic denervation of the heart by means of a left stellate ganglionectomy. Although left stellate ganglionectomy has been shown to be effective in reducing the incidence of SCD, there are complications associated with such surgery such as hemothorax and pneumothorax. In addition, there is the potential for causing permanent Horner's syndrome (eyelid drooping or ptosis, papillary constriction, and vasodilation with absence of sweating on the face and neck) resulting from the loss of sympathetic innervation.

### BRIEF DESCRIPTION OF THE DRAWINGS

FIG. 1 illustrates an exemplary neural stimulation device.

FIG. 2 is a block diagram of an implantable device for delivering neural stimulation.

FIGS. 3 and 4 illustrate different embodiments of circuitry for delivering neural stimulation pulse trains.

### DETAILED DESCRIPTION

Described herein are methods and devices for treating patients at risk for SCD by using electro-stimulation to inhibit cardiosympathetic activity. (As the terms are used herein, “electro-stimulation” or “neural stimulation” refers to electrical signals and not to the effect such electrical signals have on the tissue to which they are delivered. Depending on how it is delivered, neural stimulation may be either excitatory stimulation that elicits production of propagating action potentials or inhibitory stimulation that acts to prevent production of such action potentials.) The most important post-ganglionic neurons that conduct efferent parasympathetic signals to the heart are located in the left cervicothoracic ganglion, also sometimes called the left stellate ganglion. As described below, an implantable medical device may be equipped with a pulse generator for delivering inhibitory electrical stimulation to one or more selected sites of the left stellate ganglion via appropriately disposed neural stimulation electrodes. Such inhibitory electrical stimulation could also be delivered using neural stimulation electrodes disposed at other sites as deemed appropriate for a particular patient (e.g., the right stellate ganglion or post-ganglionic sympathetic nerve fibers). The device may be equipped with multiple neural stimulation electrodes that can be configured or not into neural stimulation channels after implantation in accordance with empirical testing. In various embodiments, inhibitory neural stimulation may be delivered chronically, intermittently per a defined schedule, in response to a patient-initiated command, or in accordance with a measured or derived physiological variable or detected physiological condition. The device may be a dedicated neural stimulation device or may be combined with a cardiac resynchronization device, ICD and/or pacemaker.

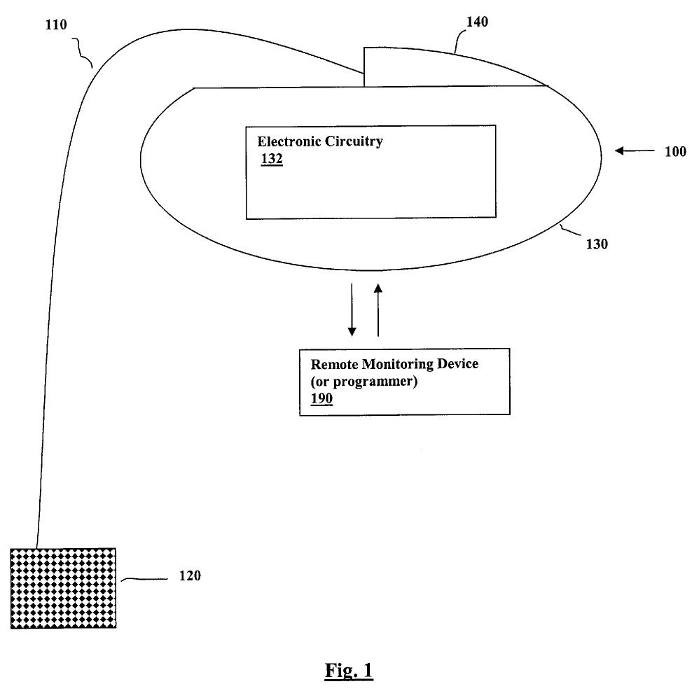

FIG. 1 shows an exemplary implantable neural stimulation device 100 that includes a hermetically sealed housing 130 that is placed subcutaneously or submuscularly in a patient's chest or other convenient location similar to a cardiac pacemaker or ICD. The housing 130 may be formed from a conductive metal, such as titanium, and may serve as an electrode for delivering electrical stimulation with a unipolar lead. Electrical pulse generation circuitry within the housing 130 is connected to a lead 110 that incorporates one or more electrodes at its distal end for stimulating nervous tissue. In one embodiment, the lead is a multi-polar lead that incorporates an array of electrodes 120 that may be implanted under the left stellate ganglion LSG. In other embodiments the electrode array may be otherwise disposed near the left stellate ganglion. One or more of the electrodes in the array may then be configured into neural stimulation channels for delivering neural stimulation that inhibits cardioparasympathetic activity.

A header 140, which may be formed of an insulating material, is mounted on the housing 130 for receiving one or more leads which are electrically connected to the circuitry within the housing. Contained within the housing 130 is the electronic circuitry 132 for providing the functionality to the device as described herein which may include a power supply, sensing circuitry, pulse generation circuitry, a programmable electronic controller for controlling the operation of the device, and a telemetry transceiver capable of communicating with an external programmer or a remote monitoring device 190. An external programmer wirelessly communicates with the device 100 and enables a clinician to receive data and modify the programming of the controller. The neural stimulation device may be configured to control the delivery of neural stimulation in an open-loop fashion upon lapsed time intervals or command inputs or in a closed-loop fashion based upon sensed physiological variables. A magnetically or tactilely actuated switch may also be provided that allows the patient to initiate or stop the delivery of neural stimulation pulses.

In the embodiment shown in FIG. 1, the lead 110 incorporates a multi-electrode array grid 120 that may be placed at a stimulation site appropriate for delivering neural stimulation that inhibits cardiosympathetic activity. For example, the grid 120 may be fixed to the stellate ganglion by suturing or otherwise during thorascopic, open or concomitant surgical procedure. The grid 120 comprises an array of individual electrodes that are spread over a relatively wide area with each electrode being connected to a separate conductor in the lead 110. After implantation of the neural stimulation device, the individual electrodes of the grid 120 may be configured into neural stimulation channels using an external programmer and tested for their efficacy in producing cardiosympathetic inhibition using different stimulation waveforms and polarity configurations. In this way, the neural stimulation channels may be configured optimally in a manner that allows for individual anatomic variation in those ganglion sites that are most important in supplying sympathetic innervation to the heart. Other embodiments may use another type of multi-electrode lead, multiple electrodes on separate leads, or a lead with a single electrode.

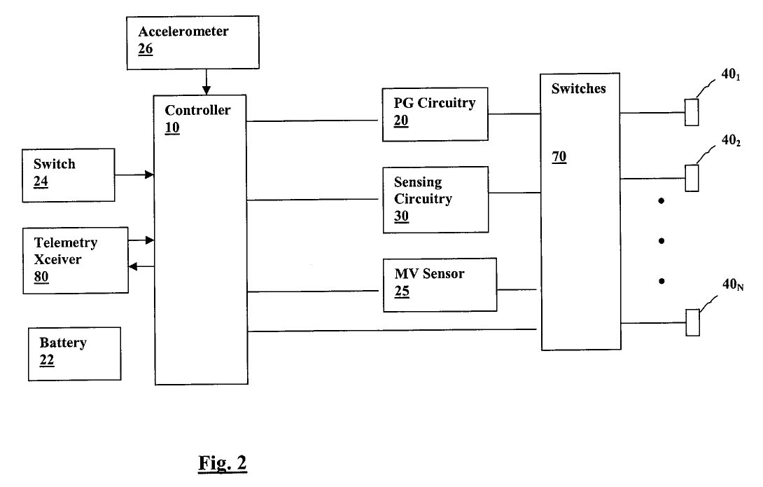

FIG. 2 is a system diagram of exemplary electronic components contained within the housing 130 of the neural stimulator. A battery 22 contained within the housing provides power to the device. A programmable electronic controller 10 is interfaced to pulse generation circuitry 20 and controls the output of neural stimulation pulses. The controller may also be interfaced to sensing circuitry for sensing cardiac activity or other physiological variables. The controller 10 may be made up of a microprocessor communicating with a memory, where the memory may comprise a ROM (read-only memory) for program storage and a RAM (random-access memory) for data storage. The controller could also be implemented by other types of logic circuitry (e.g., discrete components or programmable logic arrays) using a state machine type of design. As the term is used herein, the programming of the controller refers to either code executed by a microprocessor or to specific configurations of hardware components for performing particular functions. The controller includes circuitry for generating clock signals used to keep track of lapsed time intervals and may also be used to deliver neural stimulation in accordance with a defined schedule. A telemetry transceiver 80 is interfaced to the controller to enable communication with an external programmer or other external device and allow configuration of selected neural stimulation channels after device implantation.

The pulse generation circuitry 20 may be similar to that used in cardiac pacemakers and delivers electrical stimulation pulses through one or more neural stimulation channels, where a neural stimulation channel is made up of a pulse generator connected to an electrode. The pulse generation circuitry 20 may include capacitive discharge or current source pulse generators, registers for controlling the pulse generators, and registers for adjusting pacing parameters such as pulse energy (e.g., pulse amplitude and width), polarity, and frequency. Shown in the figure are electrodes 40₁ through 40_{N} where N is some integer. The electrodes 40₁ through 40_{N} could include, for example, the electrodes of multi-electrode grid array 120. A MOS switch matrix 70 is used to electrically connect these electrodes, as well as possibly additional electrodes on different leads, to the output of a pulse generator. The switch matrix 70 is controlled by the controller and is used to switch selected electrodes to the output of a pulse generator in order to configure a particular neural stimulation channel. The device may be equipped with any number of pulse generators and electrodes that may be combined arbitrarily to form neural stimulation channels for delivering neural stimulation to particular sites. The neural stimulation pulses may be delivered according to a predetermined schedule and/or in response to sensed conditions. A magnetically or tactilely actuated switch 24 may be provided that is interfaced to the controller 10 and allows the patient to initiate and/or stop the delivery of neural stimulation pulses. The pulse frequency, pulse width, pulse amplitude, pulse polarity, burst duration, and bipolar/unipolar stimulation configuration in this embodiment are programmable parameters, the optimal settings of which depend upon the stimulation site and type of stimulation electrode.

The device may also be equipped with different sensing modalities for sensing physiological variables and may be programmed to use these variables in controlling the delivery of neural stimulation. The device in FIG. 2 incorporates sensing circuitry 30 that includes sensing amplifiers, analog-to-digital converters for digitizing sensing signal inputs from the sensing amplifiers, and registers that can be written to for adjusting the gain and threshold values of the sensing amplifiers. The switch matrix 70 may be used to connect a particular electrode to a sensing amplifier in order to configure a sensing channel. For example, the electrodes 40₁ through 40_{N} could include electrodes disposed in the atria and/or ventricles that could be configured into cardiac sensing channels. The sensing circuitry 30 allows the device to measure heart rate and to compute parameters derived therefrom such as heart rate variability and/or the presence of a tachyarrhythmia for use in controlling the delivery of neural stimulation. Electrograms generated by cardiac sensing channels may also be used to detect myocardial ischemia. The device may also be equipped with exertion level sensing modalities that are commonly used in cardiac rhythm management devices such as a minute ventilation sensor 25 and an accelerometer 26. As described below, a measured exertion level may be used as a surrogate for sympathetic activity level and used to control the delivery of neural stimulation pulses to inhibit cardiosympathetic activity. The device may also incorporate other sensing modalities for sensing variables related to a patient's autonomic balance such as blood pressure.

As noted above, the neural stimulator may also be incorporated into an implantable cardiac rhythm management device which has cardiac pacing and/or cardioversion/defibrillation functionality. In that case, the electrodes 40₁ through 40_{N} may include one or more intra-cardiac electrodes that may be configured into sensing or pacing channels in order to sense and/or pace the atria or the ventricles in a selected pacing mode, such as conventional bradycardia pacing mode or cardiac resynchronization pacing mode. The pulse generation circuitry may also include a shock pulse generator for delivering a defibrillation/cardioversion shock via a shock electrode upon detection of a tachyarrhythmia.

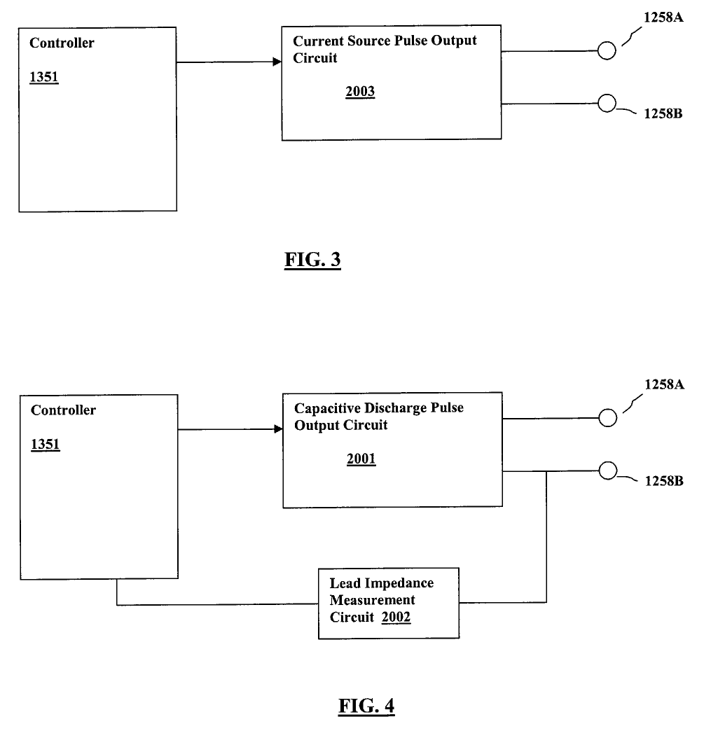

FIGS. 3 and 4 illustrate different embodiments of circuitry for delivering neural stimulation pulse trains as described above such as the pulse generators in FIG. 3 and FIG. 2. In FIG. 3, a current source pulse output circuit 2003 outputs current pulses between stimulation electrodes 1258A and 1258B in accordance with command inputs from the controller 1351. The command inputs from the controller which may be programmed by a user specify the frequency of the pulses, pulse widths, current amplitude, pulse polarity, and whether unipolar or bipolar stimulation is to be delivered. FIG. 4 illustrates another embodiment in which a capacitive discharge pulse output circuit 2001 is used to output voltage pulses between stimulation electrodes 1258A and 1258B in accordance with command inputs from the controller 1351. In this embodiment, the command inputs from the controller which may be programmed by a user specify the frequency of the pulses, pulse widths, voltage amplitude, pulse polarity, and whether unipolar or bipolar stimulation is to be delivered. In order for the controller to specify the voltage amplitude that results in the desired current amplitude for the pulses, the lead impedance may be measured by lead impedance measurement circuit 2002. The output capacitor of the pulse output circuit may then be charged to the appropriate voltage for each pulse. In order to monitor the lead impedance, the controller is programmed to periodically, or upon command from a user via telemetry, charge the output capacitor to a known voltage level, connect the output capacitor to the stimulation leads to deliver a stimulation pulse, and measure the time it takes for the capacitor voltage to decay by a certain amount (e.g., to half of the initial value).

As noted above, electrical stimulation applied to nervous tissue, depending upon the manner in which it is delivered, may either excite such tissue and generate propagating action potentials or may act as a nerve block that inhibits the generation of such action potentials. The mechanisms behind the latter phenomenon are not completely understood although it is supposed that inactivation of sodium channels and/or activation of potassium channels brought about by a depolarizing stimulus may be involved. It is also possible to produce a nerve block with a hyperpolarizing stimulus. For example, it has been demonstrated in certain situations that high frequency biphasic stimulation, delivered as either rectangular pulses or a sinusoidal waveform is effective in blocking the activity of nervous tissue. For example, in one embodiment, an implantable pulse generation device is configured to deliver inhibitory neural stimulation at frequency between 350 Hz and 12.5 kHz, while other embodiments may use higher or lower frequencies. The optimal frequency for inhibitory stimulation may vary with the individual patient due to anatomical variation of the left stellate ganglion and may also vary with the stimulation amplitude and type of waveform. Stimulation applied in the form of a hyperpolarizing DC pulse (i.e., anodic stimulation) may also be effective in producing a nerve block. After implantation of the device, the neural stimulation channels and stimulation parameters (e.g., type of waveform, amplitude, polarity, and frequency) may be configured to optimally block cardiosympathetic activity in accordance with empirical data derived from clinical testing.

In one embodiment, the neural stimulation device is configured to deliver neural stimulation for inhibiting cardiosympathetic activity on a continuous basis. In another embodiment, the neural stimulation device is configured to alternate between operating in either a neural stimulating (NS) state or a non-neural stimulating (non-NS) state. When in the NS state, the device delivers neural stimulation according to programmed stimulation parameters that inhibit cardiosympathetic activity by acting on, for example, the left stellate ganglion. In the non-NS state, the device delivers no neural stimulation. The durations of the NS and non-NS states thus define a neural stimulation duty cycle. As described below, the device may be programmed to control the duty cycle of the NS state in accordance with defined entry and exit conditions. Examples of entry and exit conditions include: a lapsed time interval, actuation of a patient-operated switch that the patient may operate, receipt of a telemetry command, detection or non-detection of the presence of myocardial ischemia by the device in accordance with a sensed variable that is correlated with the presence of myocardial ischemia such as features derived from sensed cardiac electrical activity, or a sensed physiological variable or parameter derived therefrom being below or above a specified threshold value. The device may be equipped with appropriate sensors and configured to measure physiological variables such as heart rate, PR interval, minute ventilation, activity level, blood pressure, and heart rate variability that can be compared with threshold values and used for entry and/or exit conditions. Such physiological variables used for entry and/or exit conditions could indicate the presence of a cardiac arrhythmia, be reflective of a patient's autonomic balance, and/or indicates a condition that indicates that the patient is at an increased risk of an arrhythmia and/or sudden cardiac death. Neural stimulation that inhibits cardiosympathetic activity could then be delivered when sympathetic tone is high and/or the patient is deemed to be at an increased risk of SCD. A physiological variable may be an instantaneous measurement or an average of previously measured values over some specified period of time. A physiological variable may also represent measurement of a single variable or a composite function of a plurality of variables. For example, a physiological variable could be a weighted average of heart rate, blood pressure, activity level, and respiratory rate. A composite entry and/or exit condition may also be formed by ANDing or ORing any of the conditions mentioned above in any desired manner. Entry and exit conditions thus enable the device to deliver neural stimulation for inhibiting cardiosympathetic activity upon receiving a command input to do so or when a condition is detected by the device that indicates an increased risk for a ventricular arrhythmia and/or SCD.

The methods and devices described above have been for the purpose of treating patients at risk for SCD, as either an alternative or an adjunct to other therapies such as an ICD. It should be appreciated, however, that such methods and devices may also be utilized to treat any condition characterized by an abnormal level of sympathetic nervous activity.

What is claimed is:

1. An implantable device, comprising:
a pulse generator for outputting neural stimulation pulses;
one or more stimulation electrodes connected to the pulse generator for delivering electrical stimulation to a selected neural site, wherein the stimulation electrodes are adapted for disposition near the left stellate ganglion of a patient;
a controller connected to the pulse generator for controlling the output of neural stimulation pulses in a manner that inhibits cardiosympathetic activity by acting on the left stellate ganglion;
wherein the controller is programmed to neural stimulation pulses according to a duty cycle that alternates between a neural stimulation state and a non-neural stimulation state in accordance with one or more defined entry and exit conditions;
one or more sensors connected to the controller for sensing one or more physiological variables related to an increased risk of sudden cardiac death; and,
wherein the controller is programmed to transition to the neural stimulation state and deliver neural stimulation that inhibits cardiosympathetic activity according to an entry condition defined as when the one or more physiological variables indicate that the patient is at increased risk for sudden cardiac death.

2. The device of claim 1 wherein the one or more sensors include a sensor for measuring a physiological variable selected from a group consisting of heart rate, PR interval, minute ventilation, activity level, blood pressure, myocardial ischemia, and heart rate variability.

3. The device of claim 1 further comprising a patient-operated switch and wherein the defined entry and exit conditions include receipt of a command via the patient-operated switch to enter or exit the neural stimulation state.

4. The device of claim 1 further comprising a telemetry transceiver and wherein the defined entry and exit conditions include receipt of a command via the telemetry transceiver to enter or exit the neural stimulation state.

5. The device of claim 1 wherein the defined entry and exit conditions include conditions selected from a group consisting of a lapsed time interval, detection or non-detection of the presence of myocardial ischemia by the device in accordance with a sensed variable that is correlated with the presence of myocardial ischemia such as features derived from sensed cardiac electrical activity, or a sensed physiological variable being below or above a specified threshold value.

6. The device of claim 1 wherein the one or more stimulation electrodes further comprises a grid array incorporating a plurality of stimulation electrodes connectable to the pulse generator by a switch matrix for delivering electrical stimulation to a selected neural site, wherein the grid array is adapted for disposition near the left stellate ganglion of a patient.

7. The device of claim 1 wherein the pulse generator may be adjusted to output neural stimulation pulses as biphasic pulses at a specified amplitude and frequency to optimally block cardiosympathetic activity in accordance with empirical data derived from clinical testing.

8. An implantable device, comprising:
a pulse generator for outputting neural stimulation pulses;
a grid array incorporating a plurality of stimulation electrodes connectable to the pulse generator for delivering electrical stimulation to a selected neural site, wherein the grid array is adapted for disposition near the left stellate ganglion of a patient;
a controller connected to the pulse generator for controlling the output of neural stimulation pulses in a manner that inhibits cardiosympathetic activity by acting on the left stellate ganglion;
a telemetry transceiver interfaced to the controller; and,
wherein the controller is programmed to configure neural stimulation channels with one or more electrodes of the grid array by operating a switch matrix in accordance with commands received via the telemetry transceiver.

9. The device of claim 8 wherein the switch matrix may be operated to configure one or more neural stimulation channels that optimally block cardiosympathetic activity in accordance with empirical data derived from clinical testing.

10. The device of claim 8 wherein the pulse generator may be adjusted to output neural stimulation pulses as biphasic pulses at a specified amplitude and frequency to optimally block cardiosympathetic activity in accordance with empirical data derived from clinical testing.

11. The device of claim 8 further comprising:
one or more sensors connected to the controller for sensing one or more physiological variables related to a patient's sympathetic activity; and,
wherein the controller is programmed to increase the amount of inhibitory neural stimulation if one or more physiological variables indicates that the patient's sympathetic activity is above a specified threshold value.

12. A method, comprising:
implanting a pulse generator connected to one or more neural stimulation electrodes;
implanting the one or more stimulation electrodes near the left stellate ganglion of a patient; and,
delivering neural stimulation pulses from the pulse generator to the left stellate ganglion in a manner that inhibits cardiosympathetic activity.

13. The method of claim 12 wherein the one or more stimulation electrodes is a plurality of electrodes incorporated into grid array and further comprising:
configuring different neural stimulation channels by selectively connecting electrodes of the grid array to the pulse generator via operation of a switch matrix;
testing different neural stimulation channels for efficacy in inhibiting cardiosympathetic activity by delivering neural stimulation pulses to the left stellate ganglion; and,
selecting a neural stimulation channel configured with an electrode of the grid array that optimally inhibits cardiosympathetic activity.

14. The method of claim 12 further comprising configuring the pulse generator to deliver neural stimulation for inhibiting cardiosympathetic activity on a continuous basis.

15. The method of claim 12 further comprising configuring the pulse generator to deliver neural stimulation for inhibiting cardiosympathetic activity on an intermittent basis in accordance with defined entry and exit conditions.

16. The method of claim 15 wherein the defined entry and exit conditions include conditions selected from a group consisting of a lapsed time interval, detection or non-detection of the presence of myocardial ischemia by the device in accordance with a sensed variable that is correlated with the presence of myocardial ischemia such as features derived from sensed cardiac electrical activity, or a sensed physiological variable being below or above a specified threshold value.

17. The method of claim 12 further comprising selecting a particular neural stimulation waveform for inhibiting cardiosympathetic activity in accordance with clinical testing.

18. The method of claim 12 further comprising selecting a particular neural stimulation amplitude for inhibiting cardiosympathetic activity in accordance with clinical testing.

19. The method of claim 12 further comprising selecting a particular neural stimulation frequency for inhibiting cardiosympathetic activity in accordance with clinical testing.

20. The method of claim 12 wherein the neural stimulation is delivered as biphasic pulses at a frequency between 350 Hz and 12.5 kHz.
